(19) United States
(12) Patent Application Publication
(10) Pub. No.: US 2009/0286875 A1
(43) Pub. Date: Nov. 19, 2009

(54) ISOCYSTENE DERIVATIVES FOR THE TREATMENT OF PAIN

(75) Inventor: Simon John Mantell, Sandwich (GB)

Correspondence Address: PHARMACIA CORPORATION;GLOBAL PATENT DEPARTMENT, POST OFFICE BOX 1027, ST. LOUIS, MO, 63006, US

(73) Assignee: Pfizer, Inc., New York, NY (US)

(21) Appl. No.: 12/093,475

(22) PCT Filed: Nov. 9, 2006
(86) PCT No.: PCT/IB06/03241

§ 371 Date: Sep. 5, 2008

Related U.S. Application Data
(60) Provisional application No. 60/738,150, filed on Nov. 17, 2005.

(51) Int. Cl. A61K 31/27; C07C 261/00

(52) U.S. Cl. 514/489; 560/29

(57) ABSTRACT

The present invention relates to compounds of formula (I): wherein R¹, R², R⁴ and R^{4a} are as defined herein. The invention also relates to the use of compounds of formula (I) for the treatment of pain.

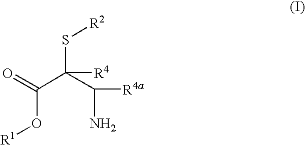

This invention relates to β-amino acid derivatives. More particularly, this invention relates to thio-substituted β-amino acid derivatives and to processes for the preparation of, intermediates used in the preparation of, compositions containing, and the uses of such derivatives.

EP-A-0347000A discusses vanilloid derivatives useful for the treatment of herpes simplex infections. WO-A-99/65881 discusses heterocyclic derivatives useful as hypoglycaemic agents. WO-A-01/56991 discusses substituted pyridine derivatives useful for controlling chemical synaptic transmission.

The compounds of the present invention are alpha-2-delta (α2δ) receptor ligands (also known as alpha-2-delta ligands) and as such are useful in the treatment of a number of different diseases. An alpha-2-delta receptor ligand is a molecule which binds to any sub-type of the human calcium channel alpha-2-delta subunit. The calcium channel alpha-2-delta subunit comprises a number of sub-types which have been described in the literature (e.g. type 1, J. Biol. Chem., 1996, 271(10), 5768-76; types 2 and 3, J. Membr. Biol., 2001, 184(1), 35-43 and Mol. Pharmacol., 2001, 59(5), 1243-1248; and type 4, Mol. Pharmacol., 2002, 62(3), 485-496). Alpha-2-delta receptor ligands are also sometimes known as GABA analogues.

Among known alpha-2-delta receptor ligands are marketed drugs such as gabapentin (sold under the trade mark Neurontin) and pregabalin (sold under the trade mark Lyrica). Gabapentin is an anti-convulsant which is marketed for the treatment of epilepsy. Pregabalin is marketed for the treatment of neuropathic pain.

There is always a need to provide new drugs, which potentially have improved properties (e.g. greater potency, greater selectivity, better absorption from the gastrointestinal tract, greater metabolic stability and more favourable pharmacokinetic properties). Other potential advantages include greater or lesser penetration of the blood-brain barrier, according to the disease targeted, lower toxicity and a decreased incidence of side-effects.

The invention therefore provides, as a first embodiment A, a compound of formula (I):

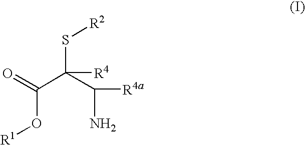

or a pharmaceutically acceptable salt or solvate thereof, wherein
R¹ is hydrogen or (C₁-C₆)alkyl;
R² is selected from
a) (C₂-C₆)alkyl optionally substituted by one or more substituents R³,
b) phenyl, naphthyl or benzyl, each substituted by one or more substituents R³, and
c) (C₃-C₈)cycloalkyl, optionally substituted by one or more substituents R³;
   each R³ is independently selected from halogen, cyano, nitro, amino, (C₁-C₆)alkyl, (C₁-C₆)alkoxy, (C₂-C₆)alkenyl, (C₂-C₈)alkynyl, hydroxy(C₁-C₆)alkyl, (C₁-C₆)alkoxy(C₁-C₆)alkyl, halo(C₁-C₆)alkyl, halo(C₁-C₆)alkoxy, halo(C₁-C₆)alkylthio, (C₁-C₆)alkylamino, (di-(C₁-C₆)alkyl)amino, amino(C₁-C₆)alkyl, (C₁-C₆)alkylamino(C₁-C₆)alkyl, (di-(C₁-C₆)alkyl)amino(C₁-C₆)alkyl, (C₁-C₆)acyl, (C₁-C₆)acyloxy, (C₁-C₆)acyloxy(C₁-C₆)alkyl, (C₁-C₆)acylamino, (C₁-C₆)alkylthio, (C₁-C₆)alkylthiocarbonyl, (C₁-C₆)alkylsulfonyl, (C₁-C₆)alkylsulfonylamino, aminosulfonyl, (C₁-C₆)alkylaminosulfonyl, (di-(C₁-C₆)alkyl)aminosulfonyl, (C₃-C₈)cycloalkyl, Het¹, phenyl and Het²;
   Het¹ is a 5- or 6-membered saturated or partially unsaturated heterocyclic group comprising one or two heteroatom ring members each independently selected from nitrogen, oxygen and sulphur, said ring nitrogen atom optionally bearing a (C₁-C₆)alkyl substituent and said ring sulphur atom optionally bearing 1 or 2 oxygen atoms;
   Het² is a 5- or 6-membered heteroaryl group comprising either (a) from 1 to 4 nitrogen atoms or (b) one oxygen or one sulphur atom and 0, 1 or 2 nitrogen atoms; and
   R⁴ and R^{4a} are independently hydrogen or methyl;
   with the proviso that the compound is not 3-amino-2-(ethylthio)-propanoic acid.

In the above definitions, halo means fluoro, chloro or bromo and is preferably fluoro or chloro. Alkyl and alkoxy groups containing the requisite number of carbon atoms can, unless otherwise specified, be straight or branched chain. Examples of alkyl include methyl, ethyl, n-propyl, i-propyl, n-butyl, i-butyl, sec-butyl and t-butyl. Examples of alkoxy include methoxy, ethoxy, n-propoxy, i-propoxy, n-butoxy, i-butoxy, sec-butoxy and t-butoxy. Examples of cycloalkyl include cyclopropyl, cyclobutyl, cyclopentyl, cyclohexyl and cycloheptyl.

In embodiment B, the invention provides a compound of formula (I), or a pharmaceutically acceptable salt thereof, wherein R², R³, R⁴ and R^{4a} are as defined above in embodiment A, and R¹ is hydrogen.

In embodiment C, the invention provides a compound of formula (I), or a pharmaceutically acceptable salt or solvate thereof, wherein R¹ is as defined above in embodiment A or embodiment B, R³, R⁴ and R^{4a} are as defined above in embodiment A, and R² is selected from
a) (C₂-C₆)alkyl optionally substituted by one or more substituents R³;
b) phenyl substituted by one or more substituents R³; and
c) (C₃-C₈)cycloalkyl, optionally substituted by one or more substituents R³.
   Most preferably, R² is (C₂-C₆)alkyl or (C₃-C₈)cycloalkyl, each optionally substituted by one or two substituents R³, or phenyl substituted by one or two substituents R³.

In embodiment D, the invention provides a compound of formula (I), or a pharmaceutically acceptable salt or solvate thereof, wherein R¹ is as defined above in embodiment A or embodiment B, R² is as defined above in embodiment A or embodiment C, R⁴ and R^{4a} are as defined above in embodiment A, and each R³ is independently selected from halogen, (C₁-C₆)alkyl, (C₁-C₆)alkoxy, halo(C₁-C₆)alkyl, halo(C₁-C₆)alkoxy, halo(C₁-C₆)alkylthio; more preferably each R³ is independently selected from halogen, (C₁-C₆)alkyl, (C₁-C₆)alkoxy, trifluoromethyl, trifluoromethoxy and trifluoromethylthio.

In embodiment E, the invention provides a compound of formula (I), or a pharmaceutically acceptable salt or solvate thereof, wherein R¹ is as defined above in embodiment A or embodiment B, R² is as defined above in embodiment A or embodiment C, R³ is as defined above in embodiment A or embodiment D, and R⁴ and R^{4a}, are hydrogen.

Specific preferred compounds according to the invention are those listed in the Examples section below and the pharmaceutically acceptable salts and solvates thereof.

Pharmaceutically acceptable salts of the compounds of formula (I) include the acid addition and base salts thereof.

Suitable acid addition salts are formed from acids which form non-toxic salts. Examples include the acetate, adipate, aspartate, benzoate, besylate, bicarbonate/carbonate, bisulphate/sulphate, borate, camsylate, citrate, cyclamate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, pyroglutamate, saccharate, stearate, succinate, tannate, tartrate, tosylate, trifluoroacetate and xinofoate salts.

Suitable base salts are formed from bases which form non-toxic salts. Examples include the aluminium, arginine, benzathine, calcium, choline, diethylamine, diolamine, glycine, lysine, magnesium, meglumine, olamine, potassium, sodium, tromethamine and zinc salts.

Hemisalts of acids and bases may also be formed, for example, hemisulphate and hemicalcium salts.

For a review on suitable salts, see Handbook of Pharmaceutical Salts: Properties, Selection, and Use by Stahl and Wermuth (Wiley-VCH, 2002).

Pharmaceutically acceptable salts of compounds of formula (I) may be prepared by one or more of three methods:
(i) by reacting the compound of formula (I) with the desired acid or base;
(ii) by removing an acid- or base-labile protecting group from a suitable precursor of the compound of formula (I) or by ring-opening a suitable cyclic precursor, for example, a lactone or lactam, using the desired acid or base; or
(iii) by converting one salt of the compound of formula (I) to another by reaction with an appropriate acid or base or by means of a suitable ion exchange column.

All three reactions are typically carried out in solution. The resulting salt may precipitate out and be collected by filtration or may be recovered by evaporation of the solvent. The degree of ionisation in the resulting salt may vary from completely ionised to almost non-ionised.

The compounds of the invention may exist in a continuum of solid states ranging from fully amorphous to fully crystalline. The term ‘amorphous’ refers to a state in which the material lacks long range order at the molecular level and, depending upon temperature, may exhibit the physical properties of a solid or a liquid. Typically such materials do not give distinctive X-ray diffraction patterns and, while exhibiting the properties of a solid, are more formally described as a liquid. Upon heating, a change from solid to liquid properties occurs which is characterised by a change of state, typically second order (‘glass transition’). The term ‘crystalline’ refers to a solid phase in which the material has a regular ordered internal structure at the molecular level and gives a distinctive X-ray diffraction pattern with defined peaks. Such materials when heated sufficiently will also exhibit the properties of a liquid, but the change from solid to liquid is characterised by a phase change, typically first order (‘melting point’).

The compounds of the invention may also exist in unsolvated and solvated forms. The term ‘solvate’ is used herein to describe a molecular complex comprising the compound of the invention and one or more pharmaceutically acceptable solvent molecules, for example, ethanol. The term ‘hydrate’ is employed when said solvent is water.

A currently accepted classification system for organic hydrates is one that defines isolated site, channel, or metal-ion coordinated hydrates—see Polymorphism in Pharmaceutical Solids by K. R. Morris (Ed. H. G. Brittain, Marcel Dekker, 1995). Isolated site hydrates are ones in which the water molecules are isolated from direct contact with each other by intervening organic molecules. In channel hydrates, the water molecules lie in lattice channels where they are next to other water molecules. In metal-ion coordinated hydrates, the water molecules are bonded to the metal ion.

When the solvent or water is tightly bound, the complex will have a well-defined stoichiometry independent of humidity. When, however, the solvent or water is weakly bound, as in channel solvates and hygroscopic compounds, the water/solvent content will be dependent on humidity and drying conditions. In such cases, non-stoichiometry will be the norm.

Also included within the scope of the invention are multi-component complexes (other than salts and solvates) wherein the drug and at least one other component are present in stoichiometric or non-stoichiometric amounts. Complexes of this type include clathrates (drug-host inclusion complexes) and co-crystals. The latter are typically defined as crystalline complexes of neutral molecular constituents which are bound together through non-covalent interactions, but could also be a complex of a neutral molecule with a salt. Co-crystals may be prepared by melt crystallisation, by recrystallisation from solvents, or by physically grinding the components together—see Chem Commun, 17, 1889-1896, by O. Almarsson and M. J. Zaworotko (2004). For a general review of multi-component complexes, see J Pharm Sci, 64 (8), 1269-1288, by Haleblian (August 1975).

The compounds of the invention may also exist in a mesomorphic state (mesophase or liquid crystal) when subjected to suitable conditions. The mesomorphic state is intermediate between the true crystalline state and the true liquid state (either melt or solution). Mesomorphism arising as the result of a change in temperature is described as ‘thermotropic’ and that resulting from the addition of a second component, such as water or another solvent, is described as ‘lyotropic’. Compounds that have the potential to form lyotropic mesophases are described as ‘amphiphilic’ and consist of molecules which possess an ionic (such as —COO⁻Na⁺, —COO⁻K⁺, or —SO₃^{-Na}⁺) or non-ionic (such as —N⁻N⁺(CH₃)₃) polar head group. For more information, see Crystals and the Polarizing Microscope by N. H. Hartshorne and A. Stuart, 4^{th} Edition (Edward Arnold, 1970).

Hereinafter all references to compounds of formula (I) include references to salts, solvates, multi-component complexes and liquid crystals thereof and to solvates, multi-component complexes and liquid crystals of salts thereof.

The compounds of the invention include compounds of formula (I) as hereinbefore defined, including all polymorphs and crystal habits thereof, prodrugs and isomers thereof (including optical, geometric and tautomeric isomers) as hereinafter defined and isotopically-labeled compounds of formula (I).

As indicated, so-called ‘prodrugs’ of the compounds of formula (I) are also within the scope of the invention. Thus certain derivatives of compounds of formula (I) which may have little or no pharmacological activity themselves can, when administered into or onto the body, be converted into compounds of formula (I) having the desired activity, for example, by hydrolytic cleavage. Such derivatives are referred to as ‘prodrugs’. Further information on the use of prodrugs may be found in Pro-drugs as Novel Delivery Systems, Vol. 14, ACS Symposium Series (T. Higuchi and W. Stella) and Bioreversible Carriers in Drug Design, Pergamon Press, 1987 (Ed. E. B. Roche, American Pharmaceutical Association).

Prodrugs in accordance with the invention can, for example, be produced by replacing appropriate functional ties present in the compounds of formula (I) with certain moieties known to those skilled in the art as ‘pro-moieties’ as described, for example, in Design of Prodrugs by H. Bundgaard (Elsevier, 1985).

Some examples of prodrugs in accordance with the invention include
(i) where the compound of formula (I) contains a carboxylic acid functionality (—COOH), an ester thereof, for example, a compound wherein the hydrogen of the carboxylic acid functionality of the compound of formula (I) is replaced by (C₁-C₈)alkyl;
(ii) where the compound of formula (I) contains an alcohol functionality (—OH), an ether thereof, for example, a compound wherein the hydrogen of the alcohol functionality of the compound of formula (I) is replaced by (C₁-C₆)alkanoyloxymethyl; and
(iii) where the compound of formula (I) contains a primary or secondary amino functionality (—NH₂ or —NHR where R≠H), an amide thereof, for example, a compound wherein, as the case may be, one or both hydrogens of the amino functionality of the compound of formula (I) is/are replaced by (C₁-C₁₀)alkanoyl.

Further examples of replacement groups in accordance with the foregoing examples and examples of other prodrug types may be found in the aforementioned references.

Moreover, certain compounds of formula (I) may themselves act as prodrugs of other compounds of formula (I).

Also included within the scope of the invention are metabolites of compounds of formula (I), that is, compounds formed in vivo upon administration of the drug. Some examples of metabolites in accordance with the invention include
(i) where the compound of formula (I) contains a methyl group, an hydroxymethyl derivative thereof (—CH₃−>—CH₂OH):
(ii) where the compound of formula (I) contains an alkoxy group, an hydroxy derivative thereof (—OR−>—OH);
(iii) where the compound of formula (I) contains a tertiary amino group, a secondary amino derivative thereof (—NR¹R²−>—NHR¹ or —NHR²);
(iv) where the compound of formula (I) contains a secondary amino group, a primary derivative thereof (—NHR¹−>—NH₂);
(v) where the compound of formula (I) contains a phenyl moiety, a phenol derivative thereof (-Ph−>-PhOH); and
(vi) where the compound of formula (I) contains a carboxamide group, a carboxylic acid derivative thereof (—CONH₂−>COOH).

Compounds of formula (I) containing one or more asymmetric carbon atoms can exist as two or more stereoisomers. Where a compound of formula (I) contains an alkenyl or alkenylene group, geometric cis/trans (or Z/E) isomers are possible. Where structural isomers are interconvertible via a low energy barrier, tautomeric isomerism (‘tautomerism’) can occur. This can take the form of proton tautomerism in compounds of formula (I) containing, for example, an imino, keto, or oxime group, or so-called valence tautomerism in compounds which contain an aromatic moiety. It follows that a single compound may exhibit more than one type of isomerism.

Included within the scope of the present invention are all stereoisomers, geometric isomers and tautomeric forms of the compounds of formula (I), including compounds exhibiting more than one type of isomerism, and mixtures of one or more thereof. Also included are acid addition or base salts wherein the counterion is optically active, for example, d-lactate or 1-lysine, or racemic, for example, dl-tartrate or dl-arginine.

Cis/trans isomers may be separated by conventional techniques well known to those skilled in the art, for example, chromatography and fractional crystallisation.

Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC).

Alternatively, the racemate (or a racemic precursor) may be reacted with a suitable optically active compound, for example, an alcohol, or, in the case where the compound of formula (I) contains an acidic or basic moiety, a base or acid such as 1-phenylethylamine or tartaric acid. The resulting diastereomeric mixture may be separated by chromatography and/or fractional crystallization and one or both of the diastereoisomers converted to the corresponding pure enantiomer(s) by means well known to a skilled person.

Chiral compounds of the invention (and chiral precursors thereof) may be obtained in enantiomerically-enriched form using chromatography, typically HPLC, on an asymmetric resin with a mobile phase consisting of a hydrocarbon, typically heptane or hexane, containing from 0 to 50% by volume of isopropanol, typically from 2% to 20%, and from 0 to 5% by volume of an alkylamine, typically 0.1% diethylamine. Concentration of the eluate affords the enriched mixture.

When any racemate crystallises, crystals of two different types are possible. The first type is the racemic compound (true racemate) referred to above wherein one homogeneous form of crystal is produced containing both enantiomers in equimolar amounts. The second type is the racemic mixture or conglomerate wherein two forms of crystal are produced in equimolar amounts each comprising a single enantiomer.

While both of the crystal forms present in a racemic mixture have identical physical properties, they may have different physical properties compared to the true racemate. Racemic mixtures may be separated by conventional techniques known to those skilled in the art—see, for example, Stereochemistry of Organic Compounds by E. L. Eliel and S. H. Wilen (Wiley, 1994).

The present invention includes all pharmaceutically acceptable isotopically-labelled compounds of formula (I) wherein one or more atoms are replaced by atoms having the same atomic number, but an atomic mass or mass number different from the atomic mass or mass number which predominates in nature.

Examples of isotopes suitable for inclusion in the compounds of the invention include isotopes of hydrogen, such as ²H and ³H, carbon, such as ¹¹C, ¹³C and ¹⁴C, chlorine, such as ³⁶Cl, fluorine, such as ¹⁸F, iodine, such as ¹²³I and ¹²⁵I, nitrogen, such as ¹³N and ¹⁵N, oxygen, such as ¹⁵O, ¹⁷O and ¹⁸O, phosphorus, such as ³²P, and sulphur, such as ³⁵S.

Certain isotopically-labelled compounds of formula (I), for example, those incorporating a radioactive isotope, are useful in drug and/or substrate tissue distribution studies. The radioactive isotopes tritium, i.e. ³H, and carbon-14, ie. ¹⁴C, are particularly useful for this purpose in view of their ease of incorporation and ready means of detection.

Substitution with heavier isotopes such as deuterium, i.e. ²H, may afford certain therapeutic advantages resulting from greater metabolic stability, for example, increased in vivo half-life or reduced dosage requirements, and hence may be preferred in some circumstances.

Substitution with positron emitting isotopes, such as ¹¹C, ¹⁸F, ¹⁵O and ¹³N, can be useful in Positron Emission Topography (PET) studies for examining substrate receptor occupancy.

Isotopically-labeled compounds of formula (I) can generally be prepared by conventional techniques known to those skilled in the art or by processes analogous to those described in the accompanying Examples and Preparations using an appropriate isotopically-labeled reagent in place of the non-labeled reagent previously employed.

Pharmaceutically acceptable solvates in accordance with the invention include those wherein the solvent of crystallization may be isotopically substituted, e.g. D₂O, d₆-acetone, d₆-DMSO.

The compounds of formula (I), being alpha-2-delta receptor ligands, are potentially useful in the treatment of a wide range of disorders. The treatment of pain, particularly neuropathic pain, is a preferred use.

Physiological pain is an important protective mechanism designed to warn of danger from potentially injurious stimuli from the external environment. The system operates through a specific set of primary sensory neurones and is activated by noxious stimuli via peripheral transducing mechanisms (see Millan, 1999, Prog. Neurobiol., 57, 1-164 for a review). These sensory fibres are known as nociceptors and are characteristically small diameter axons with slow conduction velocities. Nociceptors encode the intensity, duration and quality of noxious stimulus and by virtue of their topographically organised projection to the spinal cord, the location of the stimulus. The nociceptors are found on nociceptive nerve fibres of which there are two main types, A-delta fibres (myelinated) and C fibres (non-myelinated). The activity generated by nociceptor input is transferred, after complex processing in the dorsal horn, either directly, or via brain stem relay nuclei, to the ventrobasal thalamus and then on to the cortex, where the sensation of pain is generated.

Pain may generally be classified as acute or chronic. Acute pain begins suddenly and is short-lived (usually twelve weeks or less). It is usually associated with a specific cause such as a specific injury and is often sharp and severe. It is the kind of pain that can occur after specific injuries resulting from surgery, dental work, a strain or a sprain. Acute pain does not generally result in any persistent psychological response. In contrast, chronic pain is long-term pain, typically persisting for more than three months and leading to significant psychological and emotional problems. Common examples of chronic pain are neuropathic pain (e.g. painful diabetic neuropathy, postherpetic neuralgia), carpal tunnel syndrome, back pain, headache, cancer pain, arthritic pain and chronic post-surgical pain.

When a substantial injury occurs to body tissue, via disease or trauma, the characteristics of nociceptor activation are altered and there is sensitisation in the periphery, locally around the injury and centrally where the nociceptors terminate. These effects lead to a hightened sensation of pain. In acute pain these mechanisms can be useful, in promoting protective behaviours which may better enable repair processes to take place. The normal expectation would be that sensitivity returns to normal once the injury has healed. However, in many chronic pain states, the hypersensitivity far outlasts the healing process and is often due to nervous system injury. This injury often leads to abnormalities in sensory nerve fibres associated with maladaptation and aberrant activity (Woolf & Salter, 2000, Science, 288, 1765-1768).

Clinical pain is present when discomfort and abnormal sensitivity feature among the patient's symptoms. Patients tend to be quite heterogeneous and may present with various pain symptoms. Such symptoms include: 1) spontaneous pain which may be dull, burning, or stabbing; 2) exaggerated pain responses to noxious stimuli (hyperalgesia); and 3) pain produced by normally innocuous stimuli (allodynia—Meyer et al., 1994, Textbook of Pain, 13-44). Although patients suffering from various forms of acute and chronic pain may have similar symptoms, the underlying mechanisms may be different and may, therefore, require different treatment strategies. Pain can also therefore be divided into a number of different subtypes according to differing pathophysiology, including nociceptive, inflammatory and neuropathic pain.

Nociceptive pain is induced by tissue injury or by intense stimuli with the potential to cause injury. Pain afferents are activated by transduction of stimuli by nociceptors at the site of injury and activate neurons in the spinal cord at the level of their termination. This is then relayed up the spinal tracts to the brain where pain is perceived (Meyer et al., 1994, Textbook of Pain, 13-44). The activation of nociceptors activates two types of afferent nerve fibres. Myelinated A-delta fibres transmit rapidly and are responsible for sharp and stabbing pain sensations, whilst unmyelinated C fibres transmit at a slower rate and convey a dull or aching pain. Moderate to severe acute nociceptive pain is a prominent feature of pain from central nervous system trauma, strains/sprains, burns, myocardial infarction and acute pancreatitis, post-operative pain (pain following any type of surgical procedure), posttraumatic pain, renal colic, cancer pain and back pain. Cancer pain may be chronic pain such as tumour related pain (e.g. bone pain, headache, facial pain or visceral pain) or pain associated with cancer therapy (e.g. postchemotherapy syndrome, chronic postsurgical pain syndrome or post radiation syndrome). Cancer pain may also occur in response to chemotherapy, immunotherapy, hormonal therapy or radiotherapy. Back pain may be due to herniated or ruptured intervertabral discs or abnormalities of the lumber facet joints, sacroiliac joints, paraspinal muscles or the posterior longitudinal ligament. Back pain may resolve naturally but in some patients, where it lasts over 12 weeks, it becomes a chronic condition which can be particularly debilitating.

Neuropathic pain is currently defined as pain initiated or caused by a primary lesion or dysfunction in the nervous system. Nerve damage can be caused by trauma and disease and thus the term ‘neuropathic pain’ encompasses many disorders with diverse aetiologies. These include, but are not limited to, peripheral neuropathy, diabetic neuropathy, post herpetic neuralgia, trigeminal neuralgia, back pain, cancer neuropathy, HIV neuropathy, phantom limb pain, carpal tunnel syndrome, central post-stroke pain and pain associated with chronic alcoholism, hypothyroidism, uremia, multiple sclerosis, spinal cord injury, Parkinson's disease, epilepsy and vitamin deficiency. Neuropathic pain is pathological as it has no protective role. It is often present well after the original cause has dissipated, commonly lasting for years, significantly decreasing a patient's quality of life (Woolf and Mannion, 1999, Lancet, 353, 1959-1964). The symptoms of neuropathic pain are difficult to treat, as they are often heterogeneous even between patients with the same disease (Woolf & Decosterd, 1999, Pain Supp., 6, S141-S147; Woolf and Mannion, 1999, Lancet, 353, 1959-1964). They include spontaneous pain, which can be continuous, and paroxysmal or abnormal evoked pain, such as hyperalgesia (increased sensitivity to a noxious stimulus) and allodynia (sensitivity to a normally innocuous stimulus).

The inflammatory process is a complex series of biochemical and cellular events, activated in response to tissue injury or the presence of foreign substances, which results in swelling and pain (Levine and Taiwo, 1994, Textbook of Pain, 45-56). Arthritic pain is the most common inflammatory pain. Rheumatoid disease is one of the commonest chronic inflammatory conditions in developed countries and rheumatoid arthritis is a common cause of disability. The exact aetiology of rheumatoid arthritis is unknown, but current hypotheses suggest that both genetic and microbiological factors may be important (Grennan & Jayson, 1994, Textbook of Pain, 397-407). It has been estimated that almost 16 million Americans have symptomatic osteoarthritis (OA) or degenerative joint disease, most of whom are over 60 years of age, and this is expected to increase to 40 million as the age of the population increases, making this a public health problem of enormous magnitude (Houge & Mersfelder, 2002, Ann Pharmacother., 36, 679-686; McCarthy et al., 1994, Textbook of Pain, 387-395). Most patients with osteoarthritis seek medical attention because of the associated pain. Arthritis has a significant impact on psychosocial and physical function and is known to be the leading cause of disability in later life. Ankylosing spondylitis is also a rheumatic disease that causes arthritis of the spine and sacroiliac joints. It varies from intermittent episodes of back pain that occur throughout life to a severe chronic disease that attacks the spine, peripheral joints and other body organs.

Another type of inflammatory pain is visceral pain which includes pain associated with inflammatory bowel disease (IBD). Visceral pain is pain associated with the viscera, which encompass the organs of the abdominal cavity. These organs include the sex organs, spleen and part of the digestive system. Pain associated with the viscera can be divided into digestive visceral pain and non-digestive visceral pain. Commonly encountered gastrointestinal (GI) disorders that cause pain include functional bowel disorder (FBD) and inflammatory bowel disease (IBD). These GI disorders include a wide range of disease states that are currently only moderately controlled, including, in respect of FBD, gastro-esophageal reflux, dyspepsia, irritable bowel syndrome (IBS) and functional abdominal pain syndrome (FAPS), and, in respect of IBD, Crohn's disease, ileitis and ulcerative colitis, all of which regularly produce visceral pain. Other types of visceral pain include the pain associated with dysmenorrhea, cystitis and pancreatitis and pelvic pain.

It should be noted that some types of pain have multiple aetiologies and thus can be classified in more than one area, e.g. back pain and cancer pain have both nociceptive and neuropathic components.

Other types of pain include:
pain resulting from musculo-skeletal disorders, including myalgia, fibromyalgia, spondylitis, sero-negative (non-rheumatoid) arthropathies, non-articular rheumatism, dystrophinopathy, glycogenolysis, polymyositis and pyomyositis;
heart and vascular pain, including pain caused by angina, myocardical infarction, mitral stenosis, pericarditis, Raynaud's phenomenon, scleredoma and skeletal muscle ischemia;
head pain, such as migraine (including migraine with aura and migraine without aura), cluster headache, tension-type headache mixed headache and headache associated with vascular disorders; and
orofacial pain, including dental pain, otic pain, burning mouth syndrome and temporomandibular myofascial pain.

Apart from pain, the compounds of formula (I) are potentially useful in the treatment of any disease or condition which is treatable using an alpha-2-delta receptor ligand. Such conditions include epilepsy, gastrointestinal disorders, premature ejaculation, burning mouth syndrome, bladder disorders (such as over active bladder), faintness attacks, fibromyalgia, hypokinesia, cranial disorders, hot flashes, essential tremor, chemical dependencies and addictions, withdrawal symptoms associated with dependencies or addictions, addictive behaviours, spasticity, arthritis, inflammatory disorders (e.g. rheumatoid arthritis, osteoarthritis, psoriasis) diuresis, premenstrual syndrome, premenstrual dysphoric disorder, tinnitus, gastric damage, Down's syndrome, demyelinating diseases (e.g. multiple sclerosis and amylolateral sclerosis, cerebral vascular disorders due to acute or chronic cerebrovascular damage (e.g. cerebral infarction, subarachnoid haemorrhage or cerebral oedema), head trauma, spinal cord trauma and neuronal damage that occurs, for instance, during stroke, in cardiac bypass surgery, in incidents of intracranial haemorrhage, in perinatal asphyxia, in cardiac arrest and in status epilepticus. Alpha-2-delta receptor ligands may also be useful in the treatment of delirium, dementia and amnestic and other cognitive or neurodegenerative disorders (e.g. Parkinson's disease, Huntingtons's disease, Alzheimer's disease, senile dementia, memory disorder, vascular dementia). They may also be useful in the treatment of movement disorders such as akinesias, dyskinesias, spasticities, Tourette's syndrome, Scott syndrome, palsys, akinetic-rigid syndrome and extra-pyramidal movement disorders. They may also be useful in the treatment of sleep disorders, mood disorders, depression, depressive disorders, bipolar disorders, anxiety disorders, panic, borderline personality disorder, schizophrenia, psychotic disorders, behavioural disturbances associated with mental retardation, autistic disorder and conduct disorder.

All of the compounds of formula (I) can be prepared by conventional routes such as by the procedures described in the general methods presented below or by the specific methods described in the Examples section and the Preparations section, or by similar methods thereto. The present invention also encompasses any one or more of these processes for preparing the compounds of formula (I), in addition to any novel intermediates used therein.

In the following general methods, R¹, R², R³, R⁴ and R^{4a} are as previously defined for a compound of formula (I) unless otherwise stated.

According to a first process, compounds of formula (I) may be prepared from compounds of formula (VIII), as illustrated by Scheme 1.

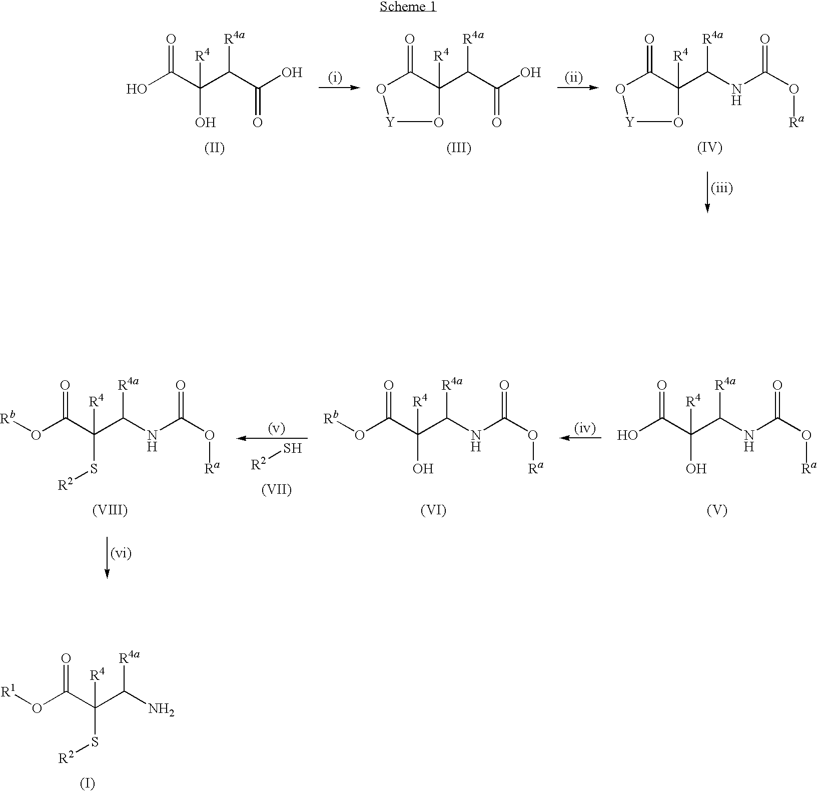

R^{a} is a suitable acid protecting group, typically (C₁-C₆)alkyl or benzyl and preferably benzyl.
R^{b} is a suitable acid protecting group, typically (C₁-C₆)alkyl
Y is a suitable 1,2-diol protecting group as described in “Protecting Groups in Organic Synthesis” by T. W. Greene and P. Wutz.

When R^{4a} is hydrogen, compounds of formula (II) are commercially available. When R⁴⁹ is not hydrogen, compounds of formula (II) may be prepared according to the methods described by Bajwa and Miller (J. Org. Chem.; 1983; 48(7); pp 1114-1116) and Eck and Simon (Tetrahedron; 1994; 50(48); pp 13641-13654).

Acetal compounds of formula (III) can be prepared from compounds of formula (II) by process step (i) using standard methodology as described in “Protecting Groups in Organic Synthesis” by T. W. Greene and P. Wutz. Typical conditions comprise the use of 1.0 equivalent of diol compound (II) and 4.0 to 4.2 equivalents of 2,2-dimethoxypropane in toluene, at a temperature of from 40° C. to 80° C. for from 1 to 8 hours.

Compounds of formula (IV) may be prepared from compounds of formula (III) by process step (ii): a compound of formula (III) is treated with a suitable azide source such as O,O-diphenylphosphoryl azide, in a suitable solvent such as toluene or xylene, at a temperature of from 80° C. to 150° C. Curtius rearrangement (Org. React. 1946, 3, 337) provides an isocyanate intermediate that is treated in situ with a suitable alcohol (R^{a}OH) to provide compounds of general formula (IV). Typical conditions comprise the use of 1.0 equivalent of a compound of formula (III) and 1.2 equivalents of O,O-diphenylphosphoryl azide in toluene, at a temperature of from 80° C. to 150° C. for from 1 to 4 hours, followed by treatment in situ with R^{a}OH, at a temperature of from room temperature to 100° C. for from 18 to 24 hours.

Compounds of formula (V) may be prepared from compounds of formula (IV) by process step (iii): hydrolysis of a compound of formula (IV), in the presence of a suitable aqueous acid such as 1M hydrochloric acid or trifluoroacetic acid, in a suitable solvent such as acetonitrile or dichloromethane, under ambient conditions for up to 18 hours. Typical conditions comprise the use of 1.0 equivalent of a compound of formula (IV) and excess dilute hydrochloric acid in acetonitrile for 18 hours at room temperature.

Compounds of general formula (VI) may be prepared from compounds of general formula (V) by process step (iv): a compound of formula (V) is treated with a suitable alkylating agent such as trimethylsilyl diazomethane, in a suitable solvent such as dichloromethane and methanol, under ambient conditions for from 1 to 18 hours. Alternatively, a compound of formula (V) may be de-protonated with a base such as sodium hydride and treated with an alkylating agent such as methyl iodide, in a suitable solvent such as acetone, at a temperature of from 20° C. to 50° C., for from 1 to 18 hours. Typical conditions comprise the use of 1.0 equivalent of a compound of formula (V) and 1.2 equivalents of trimethylsilyl diazomethane in a mixture of dichloromethane and methanol, under ambient conditions for up to 18 hours.

In a further embodiment compounds of formula (VI) can be prepared from compounds of formula (IV) by combination of process step (iii) and (iv) in a one-pot synthesis.

Compounds of formula (VII) are either commercially available or may be prepared by standard methodology known in the literature.

Compounds of formula (VIII) can be prepared from compounds of general formula (VI) by process step (v): a Mitsunobu reaction, between compounds of formulae (VI) and (VII), in the presence of a suitable phosphine compound such as tri-ⁿbutyl phosphine or triphenyl phosphine and a suitable azo compound such as diisopropylazodicarboxylate or di-tert-butyl azodicarboxylate, in a solvent such as dichloromethane, tetrahydrofuran or N,N-dimethylformamide, at temperatures of from 25° C. to 115° C., for from 1 to 48 hours. Typical conditions comprise the use of 1.0 equivalent of a compound of formula (VI), 1.0 to 1.30 equivalents of a compound of formula (VII), 1.0 to 1.3 equivalents of triphenylphosphine and 1.0 to 1.3 equivalents of di-tert-butyl azodicarboxylate, in tetrahydrofuran, under ambient conditions for 18 hours.

Compounds of formula (I) can be prepared from compounds of formula (VIII) by process step (vi): deprotection of the amino group, and optionally the carboxyl group, is achieved using standard methodology as described in “Protecting Groups in Organic Synthesis” by T. W. Greene and P. Wutz. Typical conditions comprise treatment of 1.0 equivalent of a compound of formula (VIII) with an excess of concentrated acid such as 6N hydrochloric acid, at temperatures of from room temperature to 100° C. for up to 18 hours.

According to a second process, compounds of formula (I) may be prepared from compounds of formula (X), as described in Scheme 2.

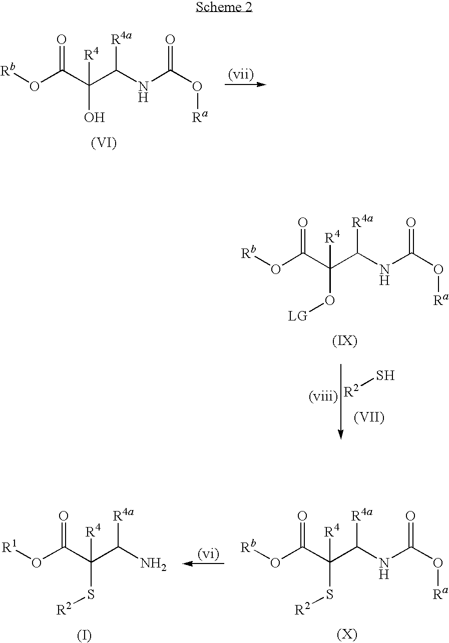

R^{a}, R^{b} and Y are as defined above for Scheme 1.

LG is a suitable leaving group such as mesylate or tosylate and is preferably mesylate.

Compounds of formula (VI) can be prepared as described for Scheme 1

Compounds of formula (IX) can be prepared from compounds of formula (VI) by process step (vii): Introduction of a suitable leaving group LG, such as mesylate or tosylate by reaction of a compound of formula (VI) with mesyl chloride/anhydride or tosyl chloride, in the presence of a suitable base such as Hünig's base, triethylamine or pyridine, in a suitable solvent such as dichloromethane or diethyl ether, at room temperature for from 1 to 6 hours. Typical conditions comprise the use of 1.0 equivalent of a compound of formula (VI), 2.0 equivalents of methanesulfonic acid anhydride and 4.0 equivalents of triethylamine in dichloromethane, under ambient conditions for from 1 to 6 hours.

Compounds of general formula (X) can be prepared from compounds of general formula (IX) and (VII) by process step (viii): a compound of formula (VII) is treated with a suitable strong base such sodium hydride or potassium tert-butoxide and the resulting anion is reacted with a compound of formula (IX), in a suitable solvent such as N,N-dimethylformamide dimethylsulfoxide or tetrahydrofuran, at a temperature of from room temperature to the reflux temperature of the solvent, for from 1 to 18 hours. Typical conditions comprise the use of 1.3 equivalents of a compound of formula (VII), 1.5 to 2.0 equivalents of sodium hydride, and 1.0 equivalent of a compound of formula (IX), in tetrahydrofuran, under ambient conditions for up to 18 hours.

Compounds of formula (I) may be prepared from compounds of formula (X) by process step (vi) as described for scheme 1.

Compounds of formula (I) may be administered as crystalline or amorphous products. They may be obtained, for example, as solid plugs, powders, or films by methods such as precipitation, crystallization, freeze drying, spray drying, or evaporative drying. Microwave or radio frequency drying may be used for this purpose.

They may be administered alone or in combination with one or more other compounds of the invention or in combination with one or more other drugs (or as any combination thereof). Generally, they will be administered as a formulation in association with one or more pharmaceutically acceptable excipients. The term ‘excipient’ is used herein to describe any ingredient other than the compound(s) of the invention. The choice of excipient will to a large extent depend on factors such as the particular mode of administration, the effect of the excipient on solubility and stability, and the nature of the dosage form.

Pharmaceutical compositions suitable for the delivery of compounds of the present invention and methods for their preparation will be readily apparent to those skilled in the art. Such compositions and methods for their preparation may be found, for example, in Remington's Pharmaceutical Sciences, 19th Edition (Mack Publishing Company, 1995).

The compounds of the invention may be administered orally. Oral administration may involve swallowing, so that the compound enters the gastrointestinal tract, and/or buccal, lingual, or sublingual administration by which the compound enters the blood stream directly from the mouth.

Formulations suitable for oral administration include solid, semi-solid and liquid systems such as tablets; soft or hard capsules containing multi- or nano-particulates, liquids, or powders; lozenges (including liquid-filled); chews; gels; fast dispersing dosage forms; films; ovules; sprays; and buccal/mucoadhesive patches.

Liquid formulations include suspensions, solutions, syrups and elixirs. Such formulations may be employed as fillers in soft or hard capsules (made, for example, from gelatin or hydroxypropylmethylcellulose) and typically comprise a carrier, for example, water, ethanol, polyethylene glycol, propylene glycol, methylcellulose, or a suitable oil, and one or more emulsifying agents and/or suspending agents. Liquid formulations may also be prepared by the reconstitution of a solid, for example, from a sachet.

The compounds of the invention may also be used in fast-dissolving, fast-disintegrating dosage forms such as those described in Expert Opinion in Therapeutic Patents, 11 (6), 981-986, by Liang and Chen (2001).

For tablet dosage forms, depending on dose, the drug may make up from 1 weight % to 80 weight % of the dosage form, more typically from 5 weight % to 60 weight % of the dosage form. In addition to the drug, tablets generally contain a disintegrant. Examples of disintegrants include sodium starch glycolate, sodium carboxymethyl cellulose, calcium carboxymethyl cellulose, croscarmellose sodium, crospovidone, polyvinylpyrrolidone, methyl cellulose, microcrystalline cellulose, lower alkyl-substituted hydroxypropyl cellulose, starch, pregelatinised starch and sodium alginate. Generally, the disintegrant will comprise from 1 weight % to 25 weight %, preferably from 5 weight % to 20 weight % of the dosage form.

Binders are generally used to impart cohesive qualities to a tablet formulation. Suitable binders include microcrystalline cellulose, gelatin, sugars, polyethylene glycol, natural and synthetic gums, polyvinylpyrrolidone, pregelatinised starch, hydroxypropyl cellulose and hydroxypropyl methylcellulose. Tablets may also contain diluents, such as lactose (monohydrate, spray-dried monohydrate, anhydrous and the like), mannitol, xylitot, dextrose, sucrose, sorbitol, microcrystalline cellulose, starch and dibasic calcium phosphate dihydrate.

Tablets may also optionally comprise surface active agents, such as sodium lauryl sulfate and polysorbate 80, and glidants such as silicon dioxide and talc. When present, surface active agents may comprise from 0.2 weight % to 5 weight % of the tablet, and glidants may comprise from 0.2 weight % to 1 weight % of the tablet.

Tablets also generally contain lubricants such as magnesium stearate, calcium stearate, zinc stearate, sodium stearyl fumarate, and mixtures of magnesium stearate with sodium lauryl sulphate. Lubricants generally comprise from 0.25 weight % to 10 weight %, preferably from 0.5 weight % to 3 weight % of the tablet.

Other possible ingredients include anti-oxidants, colourants, flavouring agents, preservatives and taste-masking agents.

Exemplary tablets contain up to about 80% drug, from about 10 weight % to about 90 weight % binder, from about 0 weight % to about 85 weight % diluent, from about 2 weight % to about 10 weight % disintegrant, and from about 0.25 weight % to about 10 weight % lubricant.

Tablet blends may be compressed directly or by roller to form tablets. Tablet blends or portions of blends may alternatively be wet-, dry-, or melt-granulated, melt congealed, or extruded before tabletting. The final formulation may comprise one or more layers and may be coated or uncoated; it may even be encapsulated.

The formulation of tablets is discussed in Pharmaceutical Dosage Forms: Tablets, Vol. 1, by H. Lieberman and L. Lachman (Marcel Dekker, New York, 1980).

Consumable oral films for human or veterinary use are typically pliable water-soluble or water-swellable thin film dosage forms which may be rapidly dissolving or mucoadhesive and typically comprise a compound of formula (I), a film-forming polymer, a binder, a solvent, a humectant, a plasticiser, a stabiliser or emulsifier, a viscosity-modifying agent and a solvent. Some components of the formulation may perform more than one function.

The compound of formula (I) may be water-soluble or insoluble. A water-soluble compound typically comprises from 1 weight % to 80 weight %, more typically from 20 weight % 0 to 50 weight %, of the solutes. Less soluble compounds may comprise a greater proportion of the composition, typically up to 88 weight % of the solutes. Alternatively, the compound of formula (I) may be in the form of multiparticulate beads.

The film-forming polymer may be selected from natural polysaccharides, proteins, or synthetic hydrocolloids and is typically present in the range 0.01 to 99 weight %, more typically in the range 30 to 80 weight %.

Other possible ingredients include anti-oxidants, colorants, flavourings and flavour enhancers, preservatives, salivary stimulating agents, cooling agents, co-solvents (including oils), emollients, bulking agents, anti-foaming agents, surfactants and taste-masking agents.

Films in accordance with the invention are typically prepared by evaporative drying of thin aqueous films coated onto a peelable backing support or paper. This may be done in a drying oven or tunnel, typically a combined coater dryer, or by freeze-drying or vacuuming.

Solid formulations for oral administration may be formulated to be immediate and/or modified release. Modified release formulations include delayed-, sustained-, pulsed-, controlled-, targeted and programmed release.

Suitable modified release formulations for the purposes of the invention are described in U.S. Pat. No. 6,106,864. Details of other suitable release technologies such as high energy dispersions and osmotic and coated particles are to be found in Pharmaceutical Technology On-line, 25(2), 1-14, by Verma et al (2001). The use of chewing gum to achieve controlled release is described in WO 00/35298.

The compounds of the invention may also be administered directly into the blood stream, into muscle, or into an internal organ. Suitable means for parenteral administration include intravenous, intraarterial, intraperitoneal, intrathecal, intraventricular, intraurethral, intrasternal, intracranial, intramuscular, intrasynovial and subcutaneous. Suitable devices for parenteral administration include needle (including microneedle) injectors, needle-free injectors and infusion techniques.

Parenteral formulations are typically aqueous solutions which may contain excipients such as salts, carbohydrates and buffering agents (preferably to a pH of from 3 to 9), but, for some applications, they may be more suitably formulated as a sterile non-aqueous solution or as a dried form to be used in conjunction with a suitable vehicle such as sterile, pyrogen-free water.

The preparation of parenteral formulations under sterile conditions, for example, by lyophilisation, may readily be accomplished using standard pharmaceutical techniques well known to those skilled in the art.

The solubility of compounds of formula (I) used in the preparation of parenteral solutions may be increased by the use of appropriate formulation techniques, such as the incorporation of solubility-enhancing agents.

Formulations for parenteral administration may be formulated to be immediate and/or modified release. Modified release formulations include delayed-, sustained-, pulsed-, controlled-, targeted and programmed release. Thus compounds of the invention may be formulated as a suspension or as a solid, semi-solid, or thixotropic liquid for administration as an implanted depot providing modified release of the active compound. Examples of such formulations include drug-coated stents and semi-solids and suspensions comprising drag-loaded poly(dl-lactic-coglycolic)acid (PGLA) microspheres.

The compounds of the invention may also be administered topically, (intra)dermally, or transdermally to the skin or mucosa. Typical formulations for this purpose include gels, hydrogels, lotions, solutions, creams, ointments, dusting powders, dressings, foams, films, skin patches, wafers, implants, sponges, fibres, bandages and microemulsions. Liposomes may also be used. Typical carriers include alcohol, water, mineral oil, liquid petrolatum, white petrolatum, glycerin, polyethylene glycol and propylene glycol. Penetration enhancers may be incorporated—see, for example, J Pharm Sci, 88 (10), 955-958, by Finnin and Morgan (October 1999).

Other means of topical administration include delivery by electroporation, iontophoresis, phonophoresis, sonophoresis and microneedle or needle-free (e.g. Powderject™, Bioject™, etc.) injection.

Formulations for topical administration may be formulated to be immediate and/or modified release. Modified release formulations include delayed-, sustained-, pulsed-, controlled-, targeted and programmed release.

The compounds of the invention can also be administered intranasally or by inhalation, typically in the form of a dry powder (either alone, as a mixture, for example, in a dry blend with lactose, or as a mixed component particle, for example, mixed with phospholipids, such as phosphatidylcholine) from a dry powder inhaler, as an aerosol spray from a pressurised container, pump, spray, atomiser (preferably an atomiser using electrohydrodynamics to produce a fine mist), or nebuliser, with or without the use of a suitable propellant, such as 1,1,1,2-tetrafluoroethane or 1,1,1,2,3,3,3-heptafluoropropane, or as nasal drops. For intranasal use, the powder may comprise a bioadhesive agent, for example, chitosan or cyclodextrin.

The pressurised container, pump, spray, atomizer, or nebuliser contains a solution or suspension of the compound(s) of the invention comprising, for example, ethanol, aqueous ethanol, or a suitable alternative agent for dispersing, solubilising, or extending release of the active, a propellant(s) as solvent and an optional surfactant, such as sorbitan trioleate, oleic acid, or an oligolactic acid.

Prior to use in a dry powder or suspension formulation, the drug product is micronised to a size suitable for delivery by inhalation (typically less than 5 microns). This may be achieved by any appropriate comminuting method, such as spiral jet milling, fluid bed jet milling, supercritical fluid processing to form nanoparticles, high pressure homogenisation, or spray drying.

Capsules (made, for example, from gelatin or hydroxypropylmethylcellulose), blisters and cartridges for use in an inhaler or insufflator may be formulated to contain a powder mix of the compound of the invention, a suitable powder base such as lactose or starch and a performance modifier such as l-leucine, mannitol, or magnesium stearate. The lactose may be anhydrous or in the form of the monohydrate, preferably the latter. Other suitable excipients include dextran, glucose, maltose, sorbitol, xylitol, fructose, sucrose and trehalose.

A suitable solution formulation for use in an atomiser using electrohydrodynamics to produce a fine mist may contain from 1 μg to 20 mg of the compound of the invention per actuation and the actuation volume may vary from 1 μl to 100 μl. A typical formulation may comprise a compound of formula (I), propylene glycol, sterile water, ethanol and sodium chloride. Alternative solvents which may be used instead of propylene glycol include glycerol and polyethylene glycol.

Suitable flavours, such as menthol and levomenthol, or sweeteners, such as saccharin or saccharin sodium, may be added to those formulations of the invention intended for inhaled/intranasal administration.

Formulations for inhaled/intranasal administration may be formulated to be immediate and/or modified release using, for example, PGLA. Modified release formulations include delayed-, sustained-, pulsed-, controlled-, targeted and programmed release.

In the case of dry powder inhalers and aerosols, the dosage unit is determined by means of a valve which delivers a metered amount. Units in accordance with the invention are typically arranged to administer a metered dose or “puff”. The overall daily dose may be administered in a single dose or, more usually, as divided doses throughout the day.

The compounds of the invention may be administered rectally or vaginally, for example, in the form of a suppository, pessary, or enema. Cocoa butter is a traditional suppository base, but various alternatives may be used as appropriate.

Formulations for rectal/vaginal administration may be formulated to be immediate and/or modified release. Modified release formulations include delayed-, sustained-, pulsed-, controlled-, targeted and programmed release.

The compounds of the invention may also be administered directly to the eye or ear, typically in the form of drops of a micronised suspension or solution in isotonic, pH-adjusted, sterile saline, Other formulations suitable for ocular and aural administration include ointments, gels, biodegradable (e.g. absorbable gel sponges, collagen) and non-biodegradable (e.g. silicone) implants, wafers, lenses and particulate or vesicular systems, such as niosomes or liposomes. A polymer such as crossed-linked polyacrylic acid, polyvinylalcohol, hyaluronic acid, a cellulosic polymer, for example, hydroxypropylmethylcellulose, hydroxyethylcellulose, or methyl cellulose, or a heteropolysaccharide polymer, for example, gelan gum, may be incorporated together with a preservative, such as benzalkonium chloride. Such formulations may also be delivered by iontophoresis.

Formulations for ocular/aural administration may be formulated to be immediate and/or modified release. Modified release formulations include delayed-, sustained-, pulsed-, controlled-, targeted, or programmed release.

The compounds of the invention may be combined with soluble macromolecular entities, such as cyclodextrin and suitable derivatives thereof or polyethylene glycol-containing polymers, in order to improve their solubility, dissolution rate, taste-masking, bioavailability and/or stability for use in any of the aforementioned modes of administration.

Drug-cyclodextrin complexes, for example, are found to be generally useful for most dosage forms and administration routes. Both inclusion and non-inclusion complexes may be used. As an alternative to direct complexation with the drug, the cyclodextrin may be used as an auxiliary additive, i.e. as a carrier, diluent, or solubiliser. Most commonly used for these purposes are alpha-, beta- and gamma-cyclodextrins, examples of which may be found in International Patent Applications Nos. WO 91/11172, WO 94/02518 and WO 98/55148.

For administration to human patients, the total daily dose of the compounds of the invention is typically in the range 1 mg to 1000 mg depending, of course, on the mode of administration. The total daily dose may be administered in single or divided doses and may, at the physician's discretion, fall outside of the typical range given herein.

These dosages are based on an average human subject having a weight of about 60 kg to 70 kg. The physician will readily be able to determine doses for subjects whose weight falls outside this range, such as infants and the elderly.

For the avoidance of doubt, references herein to “treatment” include references to curative, palliative and prophylactic treatment.

An alpha-2-delta receptor ligand may be usefully combined with another pharmacologically active compound, or with two or more other pharmacologically active compounds, particularly in the treatment of pain. For example, an alpha-2-delta receptor ligand, particularly a compound of formula (I), or a pharmaceutically acceptable salt or solvate thereof, as defined above, may be administered simultaneously, sequentially or separately in combination with one or more agents selected from:
an opioid analgesic, e.g. morphine, heroin, hydromorphone, oxymorphone, levorphanol, levallorphan, methadone, meperidine, fentanyl, cocaine, codeine, dihydrocodeine, oxycodone, hydrocodone, propoxyphene, nalmefene, nalorphine, naloxone, naltrexone, buprenorphine, butorphanol, nalbuphine or pentazocine;
a nonsteroidal antiinflammatory drug (NSAID), e.g. aspirin, diclofenac, diflusinal, etodolac, fenbufen, fenoprofen, flufenisal, flurbiprofen, ibuprofen, indomethacin, ketoprofen, ketorolac, meclofenamic acid, mefenamic acid, meloxicam, nabumetone, naproxen, nimesulide, nitroflurbiprofen, olsalazine, oxaprozin, phenylbutazone, piroxicam, sulfasalazine, sulindac, tolmetin or zomepirac;
a barbiturate sedative, e.g. amobarbital, aprobarbital, butabarbital, butabital, mephobarbital, metharbital, methohexital, pentobarbital, phenobartital, secobarbital, talbutal, theamylal or thiopental;
a benzodiazepine having a sedative action, e.g. chlordiazepoxide, clorazepate, diazepam, flurazepam, lorazepam, oxazepam, temazepam or triazolam;
an H₁ antagonist having a sedative action, e.g. diphenhydramine, pyrilamine, promethazine, chlorpheniramine or chlorcyclizine;
a sedative such as glutethimide, meprobamate, methaqualone or dichloralphenazone;
a skeletal muscle relaxant, e.g. baclofen, carisoprodol, chlorzoxazone, cyclobenzaprine, methocarbamol or orphrenadine;
an NMDA receptor antagonist, e.g. dextromethorphan ((+)-3-hydroxy-N-methylmorphinan) or its metabolite dextrorphan ((+)-3-hydroxy-N-methylmorphinan), ketamine, memantine, pyrroloquinoline quinine, cis-4-(phosphonomethyl)-2-piperidinecarboxylic acid, budipine, EN-3231 (MorphiDex®, a combination formulation of morphine and dextromethorphan), topiramate, neramexane or perzinfotel including an NR2B antagonist, e.g. ifenprodil, traxoprodil or (−)-(R)-6-{2-[4-(3-fluorophenyl)-4-hydroxy-1-piperidinyl]-1-hydroxyethyl-3,4-dihydro-2(1H)-quinolinone;
an alpha-adrenergic, e.g. doxazosin, tamsulosin, clonidine, guanfacine, dexmetatomidine, modafinil, or 4-amino-6,7-dimethoxy-2-(5-methane-sulfonamido-1,2,3,4-tetrahydroisoquinol-2-yl)-5-(2-pyridyl) quinazoline;
a tricyclic antidepressant, e.g. desipramine, imipramine, amitriptyline or nortriptyline;
an anticonvulsant, e.g. carbamazepine, lamotrigine, topiratmate or valproate;
a tachykinin (NK) antagonist, particularly an NK-3, NK-2 or NK-1 antagonist, e.g. (αR,9R)-7-[3,5-bis(trifluoromethyl)benzyl]-8,9,10,11-tetrahydro-9-methyl-5-(4-methylphenyl)-7H-[1,4]diazocino[2,1-g][1,7]-naphthyridine-6-13-dione (TAK-637), 5-[[(2R,3S)-2-[(1R)-1-[3,5-bis(trifluoromethyl)phenyl]ethoxy-3-(4-fluorophenyl)-4-morpholinyl]-methyl]-1,2-dihydro-3H-1,2,4-triazol-3-one (MK-869), aprepitant, lanepitant, dapitant or 3-[[2-methoxy-5-(trifluoromethoxy)phenyl]-methylamino]-2-phenylpiperidine (2S,3S);
a muscarinic antagonist, e.g oxybutynin, tolterodine, propiverine, tropsium chloride, darifenacin, solifenacin, temiverine and lpratropium;
a COX-2 selective inhibitor, e.g. celecoxib, rofecoxib, parecoxib, valdecoxib, deracoxib, etoricoxib, or lumiracoxib;
a coal-tar analgesic, in particular paracetamol;
a neuroleptic such as droperidol, chlorpromazine, haloperidol, perphenazine, thioridazine, mesoridazine, trifluoperazine, fluphenazine, clozapine, olanzapine, risperidone, ziprasidone, quetiapine, sertindole, aripiprazole, sonepiprazole, blonanserin, iloperidone, perospirone, raclopride, zotepine, bifeprunox, asenapine, lurasidone, amisulpride, balaperidone, palindore, eplivanserin, osanetant, rimonabant, meclinertant, Miraxion® or sarizotan;
a vanilloid receptor agonist (e.g. resinferatoxin) or antagonist (e.g. capsazepine);
a beta-adrenergic such as propranolol;
a local anaesthetic such as mexiletine;
a corticosteroid such as dexamethasone;
a 5-HT receptor agonist or antagonist, particularly a 5-HT_{1B/1D} agonist such as eletriptan, sumatriptan, naratriptan, zolmitriptan or rizatriptan;
a 5-HT_{2A} receptor antagonist such as R(+)-alpha-(2,3-dimethoxy-phenyl)-1-[2-(4-fluorophenylethyl)]-4-piperidinemethanol (MDL-100907);
a cholinergic (nicotinic) analgesic, such as ispronicline (TC-1734), (E)-N-methyl-4-(3-pyridinyl)-3-buten-1-amine (RJR-2403), (R)-5-(2-azetidinylmethoxy)-2-chloropyridine (ABT-594) or nicotine;
Tramadol®;
a PDEV inhibitor, such as 5-[2-ethoxy-5-(4-methyl-1-piperazinyl-sulphonyl)phenyl]-1-methyl-3-n-propyl-1,6-dihydro-7H-pyrazolo[4,3-d]pyrimidin-7-one (sildenafil), (6R,12aR)-2,3,6,7,12,12a-hexahydro-2-methyl-6-(3,4-methylenedioxyphenyl)-pyrazino[2′, 1′:6,1]-pyrido-[3,4]indole-1,4-dione (IC-351 or tadalafil), 2-[2-ethoxy-5-(4-ethyl-piperazin-1-yl-1-sulphonyl)-phenyl]-5-methyl-7-propyl-3H-imidazo[5,1-f][1,2,4]triazin-4-one (vardenafil), 5-(5-acetyl-2-butoxy-3-pyridinyl)-3-ethyl-2-(1-ethyl-3-azetidinyl)-2,6-dihydro-7H-pyrazolo[4,3-d]pyrimidin-7-one, 5-(5-acetyl-2-propoxy-3-pyridinyl)-3-ethyl-2-(1-isopropyl-3-azetidinyl)-2,6-dihydro-7H-pyrazolo[4,3-d]pyrimidin-7-one, 5-[2-ethoxy-5-(4-ethylpiperazin-1-ylsulphonyl)pyridin-3-yl]-3-ethyl-2-[2-methoxyethyl]-2,6-dihydro-7H-pyrazolo[4,3-d]pyrimidin-7-one, 4-[(3-chloro-4-methoxybenzyl)amino]-2-[(2S)-2-(hydroxymethyl)pyrrolidin-1-yl]-N-(pyrimidin-2-ylmethyl)pyrimidine-5-carboxamide, 3-(1-methyl-7-oxo-3-propyl-6,7-dihydro-1H-pyrazolo[4,3-d]pyrimidin-5-yl)-N-[2-(1-methylpyrrolidin-2-yl)ethyl]-4-propoxybenzenesulfonamide;
a cannabinoid;
metabotropic glutamate subtype 1 receptor (mGluR1) antagonist;
a serotonin reuptake inhibitor such as sertraline, sertraline metabolite demethylsertraline, fluoxetine, norfluoxetine (fluoxetine desmethyl metabolite), fluvoxamine, paroxetine, citalopram, citalopram metabolite desmethylcitalopram, escitalopram, d,l-fenfluramine, femoxetine, ifoxetine, cyanodothiepin, litoxetine, dapoxetine, nefazodone, cericlamine and trazodone;
a noradrenaline (norepinephrine) reuptake inhibitor, such as maprotiline, lofepramine, mirtazepine, oxaprotiline, fezolamine, tomoxetine, mianserin, buproprion, buproprion metabolite hydroxybuproprion, nomifensine and viloxazine (Vivalan®), especially a selective noradrenaline reuptake inhibitor such as reboxetine, in particular (S,S)-reboxetine;
a dual serotonin-noradrenaline reuptake inhibitor, such as venlafaxine, venlafaxine metabolite O-desmethylvenlafaxine, clomipramine, clomipramine metabolite desmethylclomipramine, duloxetine, milnacipran and imipramine;
an inducible nitric oxide synthase (iNOS) inhibitor such as S-[2-[(1-iminoethyl)amino]ethyl]-L-homocysteine, S-[2-[(1-iminoethyl)-amino]ethyl]-4,4-dioxo-L-cysteine, S-[2-[(1-iminoethyl)amino]ethyl]-2-methyl-L-cysteine, (2S,5Z)-2-amino-2-methyl-7-[(1-iminoethyl)amino]-5-heptenoic acid, 2-[[(1R,3S)-3-amino-4-hydroxy-1-(5-thiazolyl)-butyl]thio]-5-chloro-3-pyridinecarbonitrile; 2-[[(1R,3S)-3-amino-4-hydroxy-1-(5-thiazolyl)butyl]thio]-4-chlorobenzonitrile, (2S,4R)-2-amino-4-[[2-chloro-5-(trifluoromethyl)phenyl]thio]-5-thiazolebutanol, 2-[[(1R,3S)-3-amino-4-hydroxy-1-(5-thiazolyl) butyl]thio]-6-(trifluoromethyl)-3 pyridinecarbonitrile, 2-[[(1R,3S)-3-amino-4-hydroxy-1-(5-thiazolyl)butyl]thio]-5-chlorobenzonitrile, N-[4-[2-(3-chlorobenzylamino)ethyl]phenyl]thiophene-2-carboxamidine, or guanidinoethyldisulfide;
an acetylcholinesterase inhibitor such as donepezil;
a prostaglandin E₂ subtype 4 (EP4) antagonist such as N-[({2-[4-(2-ethyl-4,6-dimethyl-1H-imidazo[4,5-c]pyridin-1-yl)phenyl]ethyl}amino)-carbonyl]-4-methylbenzenesulfonamide or 4-[(1S)-1-({[5-chloro-2-(3-fluorophenoxy)pyridin-3-yl]carbonyl}amino)ethyl]benzoic acid;
a leukotriene B4 antagonist; such as 1-(3-biphenyl-4-ylmethyl-4-hydroxy-chroman-7-yl)-cyclopentanecarboxylic acid (CP-105696), 5-[2-(2-Carboxyethyl)-3-[6-(4-methoxyphenyl)-5E-hexenyl]oxyphenoxy]-valeric acid (ONO-4057) or DPC-11870,
a 5-lipoxygenase inhibitor, such as zileuton, 6-[(3-fluoro-5-[4-methoxy-3,4,5,6-tetrahydro-2H-pyran-4-yl])phenoxy-methyl]-1-methyl-2-quinolone (ZD-2138), or 2,3,5-trimethyl-6-(3-pyridylmethyl), 1,4-benzoquinone (CV-6504);
a sodium channel blocker, such as lidocaine;
a 5-HT3 antagonist, such as ondansetron;
   and the pharmaceutically acceptable salts and solvates thereof.

Where a combination of active compounds is to be administered, two or more pharmaceutical compositions may conveniently be combined in the form of a kit suitable for co-administration of the compositions. Such a kit comprises two or more separate pharmaceutical compositions, at least one of which contains an alpha-2-delta receptor ligand, particularly a compound of formula (I), and means for separately retaining said compositions, such as a container, divided bottle, or divided foil packet. An example of such a kit is the familiar blister pack used for the packaging of tablets, capsules and the like.

Such a kit is particularly suitable for administering different dosage forms, for example oral and parenteral formulations, for administering separate compositions at different dosage intervals, or for titrating separate compositions against one another. To assist compliance, the kit typically comprises directions for administration and may be provided with a so-called memory aid.

It will be appreciated that what the invention provides, and what will be claimed, is as follows;

(i) a compound of formula (I) or a pharmaceutically acceptable salt or solvate thereof;
(ii) a compound of formula (I) or a pharmaceutically acceptable salt or solvate thereof, for use as a medicament;
(ii) the use of a compound of formula (I) or a pharmaceutically acceptable salt or solvate thereof, for the manufacture of a medicament for the treatment of a disease for which an alpha-2-delta receptor ligand is indicated;
(iii) the use of a compound of formula (I) or a pharmaceutically acceptable salt or solvate thereof, for the manufacture of a medicament for the treatment of pain;
(iv) a method of treating a disease or condition for which an alpha-2-delta receptor ligand is indicated in a mammal, including a human being, comprising administering an effective amount of a compound of formula (I) or a pharmaceutically acceptable salt or solvate thereof;
(v) a method of treating pain in a mammal, including a human being, comprising administering an effective amount of a compound of formula (I) or a pharmaceutically acceptable salt or solvate thereof;
(vi) a pharmaceutical composition including a compound of formula (I) or a pharmaceutically acceptable salt or solvate thereof, together with a pharmaceutically acceptable excipient;
(vii) a process for the preparation of a compound of formula (I) or a pharmaceutically acceptable salt or solvate thereof;
(viii) certain novel intermediates disclosed herewithin; and
(ix) a combination of a compound of formula (I) and one or more further pharmacologically active compounds.

The following Examples illustrate the preparation of the compounds of formula (I).

¹H Nuclear magnetic resonance (NMR) spectra were in all cases consistent with the proposed structures. Characteristic chemical shifts (δ) are given in parts-per-million downfield from tetramethylsilane using conventional abbreviations for designation of major peaks: e.g. s, singlet; d, doublet; t, triplet; q, quartet; m, multiplet; br, broad. The mass spectra (MS) were recorded using either electrospray ionisation (ESI) or atmospheric pressure chemical ionisation (APCI). The following abbreviations have been used for common solvents: CDCl₃, deuterochloroform; D₆-DMSO, deuterodimethylsulphoxide; CD₃OD, deuteromethanol; THF, tetrahydrofuran. LCMS indicates liquid chromatography mass spectrometry (R₁=retention time).

### EXAMPLE 1

### 3-Amino-2-[(1-ethylpropyl)thio]propanoic acid hydrochloride

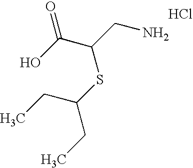

A mixture of the product of Preparation 7 (49 mg, 0.14 mmol) and 6N hydrochloric acid (15 mL) was heated under reflux for 18 hours. The reaction mixture was then cooled to room temperature, washed with dichloromethane (4×20 mL) and concentrated in vacuo to afford the title compound as a white solid in 46% yield, 15 mg

¹HNMR (400 MHz, CD₃OD) δ: 0.99-1.02 (m, 6H), 1.55-1.80 (m, 4H), 2.84-2.81 (m, 1H), 3.15-3.20 (m, 1H), 3.31-3.36 (m, 1H), 3.58-3.62 (m, 1H); LRMS ESI m/z 192 [M+H]⁺

### EXAMPLES 2 TO 8

The following compounds, of the general formula shown below, were prepared from the products of Preparations 5 and 8 to 13, using the same method as described for Example 1.

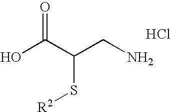

| No. | R² | Data | Yield |
|---|---|---|---|
| 2 | 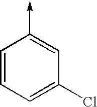 | ¹HNMR(400 MHz, CD₃OD) δ: 3.17-3.28(m, 2 H), 3.9- 4.00(m, 1 H), 7.36-7.37(m, 2 H), 7.48-7.51(m, 1 H), 7.61- 7.62(m, 1 H); LRMS ESI m/z 232 [M + H]⁺ | 80% |
| 3 | —CH(CH₃)₂ | ¹HNMR(400 MHz, CD₃OD) δ: 1.32-1.35(m, 6 H), 3.14- | 22% |
| | | 3.40(m, 3 H), 3.67-3.71(m, 1 H); LRMS ESI m/z 164 | |
| | | [M + H]⁺ | |
| 4 | —C(CH₃)₃ | ¹HNMR(400 MHz, CD₃OD) δ: 1.40(s, 9 H), 3.15-3.19(m, | 20% |
| | | 1 H), 3.26-3.33(m, 1 H), 3.61-3.67(m, 1 H); LCMS mz 178 | |
| | | [M + H]⁺ | |
| 5 | 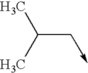 | ¹HNMR(400 MHz, CD₃OD) δ: 1.00-1.02(m, 6 H), 1.80- 1.90(m, 1 H), 2.60-2.70(m, 2 H), 3.15-3.25(m, 1 H), 3.30- 3.40(m, 1 H), 3.60(m, 1 H); LRMS ESI m/z 178 [M + H]⁺ | 91% |
| 6 | 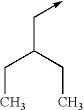 | ¹HNMR(400 MHz, CD₃OD) δ: 0.88-0.92(m, 6 H), 1.39- 1.52(m, 5 H), 2.65-2.80(m, 2 H), 3.15-3.22(m, 1 H), 3.33- 3.40(m, 1 H), 3.55-3.59(m, 1 H); LRMS ESI m/z 206 [M + H]⁺ | 6% |
| 7 | 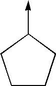 | ¹HNMR(400 MHz, CD₃OD) δ: 1.51-1.67(m, 4 H), 1.71- 1.82(m, 2 H), 2.04-2.15(m, 2 H), 3.17-3.22(m, 1 H), 3.00- 3.43(m, 2 H), 3.64-3.66(m, 1 H); LRMS ESI m/z 190 [M + H]⁺ | 30% |
| 8 | 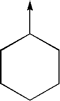 | ¹HNMR(400 MHz, CDCl₃) δ: 1.23-1.44(m, 5 H), 1.59- 1.67(m, 1 H) ,1.73-1.85(m, 2 H), 1.97-2.10(m, 2 H), 2.95- 3.04(m, 1 H), 3.14-3.19(m, 1 H), 3.30-3.36(m, 1 H), 3.65- 3.69(m, 1 H); LRMS ESI m/z 204 [M + H]⁺ | 28% |

### Preparation 1

### [(4R)-2,2-Dimethyl-5-oxo-1,3-dioxolan-4-yl]acetic acid

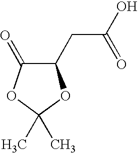

2,2-Dimethoxypropane (75 mL, 610 mmol) was added to a solution of D(+)malic acid (19.8 g, 148 mmol) in toluene (200 mL) and the mixture was heated under reflux for 2.5 hours. The reaction mixture was concentrated in vacuo and the residue was purified by column chromatography on silica gel, eluting with diethyl ether. The appropriate fractions were evaporated under reduced pressure and the residue was further purified by column chromatography on silica gel, eluting with heptane:diethyl ether, 66:33 to 50:50 to 0:100, to afford the title compound in 79% yield, 20.2 g.

¹HNMR (400 MHz, CDCl₃) δ: 1.58 (s, 3H), 1.63 (s, 3H), 2.80-3.05 (m, 2H), 4.70 (m, 1H)

### Preparation 2

Benzyl {[(4R)-2,2-dimethyl-5-oxo-1,3-dioxolan-4-yl]methyl}carbamate

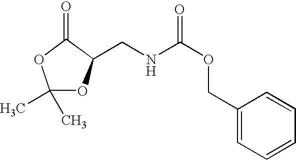

A mixture of the product of Preparation 1 (9.8 g, 56 mmol), triethylamine (9.41 mL, 68 mmol) and O,O-diphenylphosphoryl azide (13.34 mL, 61.6 mmol) in toluene (100 mL) was heated at 85° C. for 90 minutes. Benzyl alcohol (6.43 mL, 62 mmol) was then added and heating continued at 85° C. for 20 hours. The cooled reaction mixture was concentrated in vacuo and the residue was partitioned between dichloromethane (200 mL) and water (100 mL). The organic layer was separated, washed with brine (100 mL), and the combined aqueous solution was extracted with dichloromethane (200 mL). The combined organic solution was washed with sodium hydrogen carbonate solution, dried over magnesium sulfate and concentrated in vacuo. The residue was purified by column chromatography on silica gel, eluting with dichloromethane. The appropriate fractions were evaporated under reduced pressure and the residue was further purified by column chromatography on silica gel, eluting with heptane:ethyl acetate, 75:25 to 66:33, to afford the title compound as an oil in 35% yield, 5.49 g.

¹HNMR (400 MHz, CDCl₃) δ: 1.54 (s, 3H), 1.56 (s, 3H), 3.55-3.72 (m, 2H), 4.46-4.49 (m, 1H), 5.05-5.16 (m, 2H), 7.31-7.36 (m, 5H); LCMS m/z 280 [M+H]⁺

### Preparation 3

### (2R)-3-{[(Benzyloxy)carbonyl]amino}-2-hydroxypropanoic acid

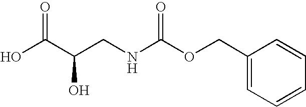

A solution of the product of Preparation 2 (4.29 g, 15 mmol) in acetonitrile (80 mL) was treated with 1M hydrochloric acid (80 mL) and the mixture was stirred at room temperature for 18 hours. The reaction mixture was then concentrated in vacuo to low volume and mixture was filtered, washing through with water. The residue was dissolved in acetone (100 mL) and the solution was stirred for 5 minutes and filtered. The filtrate was diluted with toluene and concentrated in vacuo to afford the title compound as a white solid in 83% yield, 2.97 g.

¹HNMR (400 MHz, CD₃OD) δ: 3.33-3.40 (m, 1H), 3.50-3.55 (m, 1H), 4.20-4.23 (m, 1H), 5.08 (s, 2H), 7.27-7,34(m, 5H); LCMS m/z 238 [M+H]⁺

### Preparation 4

### Methyl (2R)-3-{[(benzyloxy)carbonyl]amino}-2-hydroxypropanoate

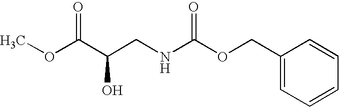

Trimethylsilyl diazomethane (2M in hexane, 39.57 mL, 79.14 mmol) was added to a solution of the product of Preparation 3 (15.78 g, 65-65 mmol) in dichloromethane (480 mL) and methanol (120 mL) and the mixture was stirred at room temperature for 18 hours. The reaction mixture was then concentrated in vacuo and the residue was dissolved in ethyl acetate (200 mL), washed with dilute acetic acid solution (200 mL) and brine (200 mL), dried over magnesium sulfate and concentrated in vacuo. Purification of the residue by column chromatography on silica gel, eluting with heptane:ethyl acetate, 50:50 to 0:100, afforded the title compound as a yellow solid in 77% yield, 12.9 g.

¹HNMR (400 MHz, CDCl₃) δ: 3.50-3.62 (m, 2H), 3.77 (s, 3H), 4.27-4.30 (m, 1H), 5.09 (s, 2H), 5.18-5.27 (m, 1H), 7.29-7.35 (m, 5H); LCMS m/z 254 [M+H]⁺

### Preparation 5

### Methyl (2S)-3-{[(benzyloxy)carbonyl]amino}-2-[(3-chlorophenyl)thio]propanoate

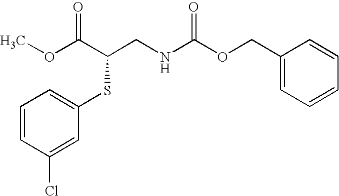

di-Isopropyl azodicarboxylate (319 mg, 1.58 mmol) was added to an ice cold solution of triphenylphosphine (414 mg, 1.58 mmol) in tetrahydrofuran (4 mL) and the mixture was stirred at room temperature for 30 minutes. The product of Preparation 4 (200 mg, 0.79 mmol) and 3-chlorobenzenethiol (119 μL, 1.03 mmol) were then added and mixture was stirred at room temperature for 48 hours. The reaction mixture was then quenched with water (few drops) and concentrated in vacuo. The residue was dissolved in ethyl acetate, washed with 1M sodium hydroxide solution (10 mL) and brine (10 mL), dried over magnesium sulfate and concentrated in vacuo. Purification of the residue by column chromatography on silica gel, eluting with heptane:ethyl acetate, 83:17, 75:25 afforded the title compound as a white solid in 34% yield, 102 mg.

¹HNMR (400 MHz, CDCl₃) δ: 3.43-3.58 (m, 2H), 3.64 (s, 3H), 3.81-3.84 (m, 1H), 5.01 (s, 2H), 5.09-5.18 (m, 1H), 7.16-29(m, 8H), 7.39 (s, 1H); LCMS m/z 380 [M+H]⁺

### Preparation 6

### Methyl (2R)-3-{[(benzyloxy)carbonyl]amino}-2-[(methylsulfonyl)oxy]propanoate

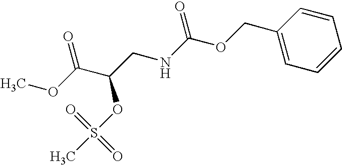

Methanesulfonic acid anhydride (2.53 g, 14.5 mmol) was added to an ice-cold solution of the product of Preparation 4 (1.84 g, 7.3 mmol) and triethylamine (4.05 mL, 29 mmol) in dichloromethane (20 mL) and the mixture was stirred at room temperature for 3 hours. The reaction mixture was then partitioned between ethyl acetate (80 mL) and water (40 mL) and the organic layer was separated, dried over magnesium sulfate and concentrated in vacuo. Purification of the residue by column chromatography on silica gel, eluting with heptane:ethyl acetate, 66:33 to 50:50, afforded the title compound in 86% yield, 2.07 g.

¹HNMR (400 MHz, CDCl₃) δ: 3.12 (s, 3H), 3.60-3.67 (m, 1H), 3.75-3.82 (m, 1H), 3.78 (s, 3H), 5.10 (s, 2H), 5.13-5.15 (m, 1H), 5.24-5.30 (m, 1H), 7.29-7.37 (m, 5H); LCMS m/z 354 [M+H]⁺

### Preparation 7

### Methyl 3-{[(benzyloxy)carbonyl]amino}-2-[(1-ethylpropyl)thio]propanoate

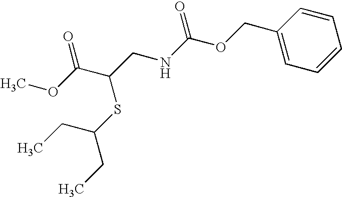

Sodium hydride (60% dispersion in mineral oil, 25 mg, 1.04 mmol) was added to a solution of the product of Preparation 14 (82 mg, 0.78 mmol) in tetrahydrofuran (4 mL) and the mixture was stirred at room temperature until effervescence had ceased. The reaction mixture was then cooled to 0° C. and a solution of the product of Preparation 6 (200 mg, 0.6 mmol) in tetrahydrofuran (1 mL) was then added and the mixture was stirred at room temperature for 90 minutes. The mixture was then partitioned between dichloromethane (40 mL) and water (40 mL) and the organic layer was separated, dried over magnesium sulfate and concentrated in vacuo. The residue was purified by column chromatography using an Isolute silica cartridge, eluting with pentane:dichloromethane, 33:66 to 0:100, to afford the title compound in 24% yield, 49 mg.

¹HNMR (400 MHz, CDCl₃) δ: 0.94-0.98 (m, 6H), 1.51-1.67 (m, 4H), 2.67-2.73 (m, 1H), 3.47-3.54 (m, 3H), 3.73 (s, 3H), 5.05-5.14 (m, 2H), 5.20-5.28 (m, 1H), 7.30-7.36 (m, S5H); LRMS ESI m/z 362 [M+Na]⁺

### Preparations 8 to 13

The following compounds, of the general formula shown below, were prepared using the same method to that described for Preparation 7, using the product of preparation 6 with the appropriate thiol starting material (either commercially available or prepared as described below).

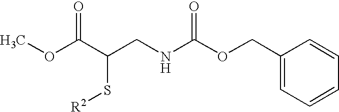

| No. | R² | Data | Yield |
|---|---|---|---|
| 8 | C(CH₃)₃ | ¹HNMR(400 MHz, CDCl₃): δ: 1.37(s, 9 H), 3.46-3.56(m, | 44% |
| | | 3 H), 3.73(s, 3 H), 5.05-5.15(m, 2 H), 5.22-5.28(m, 1 H), | |
| | | 7.30-7.36(m, 5 H); LCMS m/z 326 [M + H]⁺ | |
| 9 | 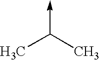 | ¹HNMR(400 MHz, CDCl₃) δ: 1.27-1.31(m, 6 H), 3.08- 3.15(m, 1 H), 3.50-3.57(m, 3 H), 3.73(s, 3 H), 5.05- 5.14(m ,2 H), 5.25-5.28(m, 1 H), 7.29-7.36(m, 5 H); LCMS m/z 312 [M + H]⁺ | quant |
| 10 | 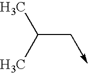 | ¹HNMR(400 MHz, CDCl₃) δ: 0.90-0.91(m, 6 H), 1.67- 1.75(m, 1 H), 2.44-2.47(m, 2 H), 3.38-3.49(m, 3 H), 3.67(s, 3 H), 4.99-5.07(m, 2 H), 5.12-5.18(m, 1 H), 7.23- 7.29(m, 5 H); LRMS ESI m/z 362 [M + Na]⁺ | 76% |
| 11 | 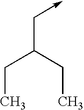 | ¹HNMR(400 MHz, CDCl₃) δ: 0.84-0.86(m, 6 H), 1.34- 1.43(m, 5 H), 2.61-2.62(m, 2 H), 3.44-3.55(m, 3 H), 3.75(s, 3 H), 5.06-5.13(m, 2 H), 5.19-5.25(m, 1 H), 7.30- 7.36(m, 5 H); LRMS ESI m/z 376 [M + Na]⁺ Starting thiol prepared in preparation 15. | 21% |
| 12 | 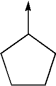 | ¹HNMR(400 MHz, CDCl₃) δ: 1.43-1.62(m, 4 H), 1.67- 1.79(m, 2 H), 1.97-2.09(m, 2 H), 3.20-3.30(m, 1 H), 3.50- 3.59(m, 3 H), 3.75(s, 3 H), 5.06-5.14(m, 2 H), 5.23(bs, 1 H), 7.29-7.38(m, 5 H); LRMS ESI m/z 338 [M + H]⁺ | 55% |
| 13 | 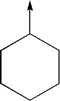 | ¹HNMR(400 MHz, CDCl₃) δ: 1.19-1.39(m, 5 H), 1.54- 1.68(m, 1 H), 1.70-1.79(m, 2 H), 1.92-2.03(m, 2 H), 2.81- 2.89(m, 1 H), 3.48-3.58(m, 3 H), 3.74(s, 3 H), 5.06- 5.14(m, 2 H), 5.23(bs, 1 H), 7.29-7.38(m, 5 H); LRMS ESI m/z 352 [M + H]⁺ | 95% |

### Preparation 14

### Pentane-3-thiol

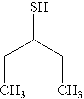

Thiourea (3.03 g, 40 mmol) was added to a solution of 3-bromopentane (5 g, 33 mmol) in ethanol (40 mL) and the mixture was heated under reflux for 2 hours. The reaction mixture was then cooled to room temperature and concentrated in vacuo. The residue was dissolved in water (30 mL), 5M sodium hydroxide solution (15 mL, 75 mmol) was added and the mixture was stirred at room temperature for 18 hours, The reaction mixture was acidified with 15% sulfuric acid and partitioned between pentane (100 mL) and water (50 mL). The organic layer was separated, dried over magnesium sulfate and concentrated in vacuo to afford the title compound in 5% yield, 200 mg.

¹HNMR (400 MHz, CDCl₃) δ: 0.97-1.01 (m, 6H), 1.30-1.32 (m, 1H), 1.44-1.55 (m, 2H), 1.62-1.74 (m, 2H), 2.62-2.70 (m, 1H)

### Preparation 15

### 2-Ethylbutane-1-thiol

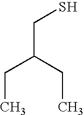

The title compound was prepared from 1-bromo-2-ethylbutane and thiourea, using the same method as that described for preparation 14, as an oil in 38% yield, 1.36 g.

¹HNMR (400 MHz, CDCl₃) δ: 0.84-0.88 (m, 6H), 1.15-1.19 (m, 1H), 1.34-1.43 (m, 5H), 2.51-2.55 (m, 2H).

The biological activity of the alpha-2-delta ligands of the invention may be measured in a radioligand binding assay using [³H]gabapentin and the α₂δ subunit derived from porcine brain tissue (Gee N. S., Brown J. P., Dissanayake V. U. K., Offord J., Thurlow R., Woodruff G. N., J. Biol. Chem., 1996; 271:5768-5776). Results may be expressed in terms of μM or nM α2δ binding affinity.

All the Examples described above Were tested in this alpha-2-delta assay and were found to have a binding affinity (IC₅₀) of 140 nM or less.

| Example Number | IC₅₀ value (nM) |
|---|---|
| 1 | 19 |
| 2 | 79 |
| 3 | 69 |
| 4 | 45 |
| 5 | 127 |
| 6 | 53 |
| 7 | 140 |
| 8 | 60 |

1. A compound of formula I: 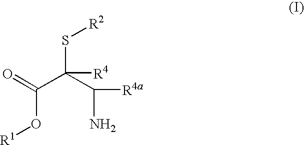
or a pharmaceutically acceptable salt or solvate thereof, wherein
R¹ is hydrogen or (C₁-C₆)alkyl;
R² is selected from
a) (C₂-C₆)alkyl optionally substituted by one or more substituents R³,
b) phenyl, naphthyl or benzyl, each substituted by one or more substituents R³, and
c) (C₃-C₈)cycloalkyl, optionally substituted by one or more substituents R³;
each R³ is independently selected from halogen, cyano, nitro, amino, (C₁-C₆)alkyl, (C₁-C₆)alkoxy, (C₂-C₆)alkenyl, (C₂-C₆)alkynyl, hydroxy(C₁-C₆)alkyl, (C₁-C₆)alkoxy(C₁-C₆)alkyl, halo(C₁-C₆)alkyl, halo(C₁-C₆)alkoxy, halo(C₁-C₆)alkylthio, (C₁-C₆)alkylamino, (di-(C₁-C₆)alkyl)amino, amino(C₁-C₆)alkyl, (C₁-C₆)alkylamino(C₁-C₆)alkyl, (di-(C₁-C₆)alkyl)amino(C₁-C₆)alkyl, (C₁-C₆)acyl, (C₁-C₆)acyloxy, (C₁-C₆)acyloxy(C₁-C₆)alkyl, (C₁-C₆)acylamino, (C₁-C₆)alkylthio, (C₁-C₆)alkylthiocarbonyl, (C₁-C₆)alkylsulfonyl, (C₁-C₆)alkylsulfonylamino, aminosulfonyl, (C₁-C₆)alkylaminosulfonyl, (di-(C₁-C₆)alkyl)aminosulfonyl, (C₃-C₈)cycloalkyl, Het¹, phenyl and Het²;
Het¹ is a 5- or 6-membered saturated or partially unsaturated heterocyclic group comprising one or two heteroatom ring members each independently selected from nitrogen, oxygen and sulphur, said ring nitrogen atom optionally bearing a (C₁-C₆)alkyl substituent and said ring sulphur atom optionally bearing 1 or 2 oxygen atoms;
Het² is a 5- or 6-membered heteroaryl group comprising either (a) from 1 to 4 nitrogen atoms or (b) one oxygen or one sulphur atom and 0, 1 or 2 nitrogen atoms; and
R⁴ and R^{4a} are independently hydrogen or methyl;
with the proviso that the compound is not 3-amino-2-(ethylthio)-propanoic acid or S-trityl-DL-isocysteine.

2. A compound of formula (I) according to claim 1 wherein R² is selected from
a) (C₂-C₆)alkyl optionally substituted by one or more substituents R³;
b) phenyl substituted by one or more substituents R³; and
c) (C₃-C₈)cycloalkyl, optionally substituted by one or more substituents R³.

3. A compound of formula (I) according to claim 1 wherein each R³ is independently selected from halogen, (C₁-C₆)alkyl, (C₁-C₆)alkoxy, halo(C₁-C₆)alkyl, halo(C₁-C₆)alkoxy and halo(C₁-C₆)alkylthio.

4. A compound of formula (I) according to claim 1 wherein R¹ is hydrogen.

5. A compound formula (I) according to claim 1 wherein R⁴ and R^{4a} are both hydrogen.

6. A compound formula (I) according to claim 1, selected from:
(2S)-3-Amino-2-[(1-ethylpropyl)thio]propanoic acid;
(2S)-3-Amino-2-[(3-chlorophenyl)thio]propanoic acid;
(2S)-3-Amino-2-[(1-methylethyl)thio]propanoic acid;
(2S)-3-Amino-2-[(t-butyl)thio]propanoic acid;
(2S)-3-Amino-2-[(2-methylpropyl)thio]propanoic acid;
(2S)-3-Amino-2-[(2-ethylbutyl)thio]propanoic acid;
(2S)-3-Amino-2-[(cyclopentyl)thio]propanoic acid;
(2S)-3-Amino-2-[(cyclohexyl)thio]propanoic acid;
and the pharmaceutically acceptable salts and solvates thereof.

7. (canceled)

8. (canceled)

9. (canceled)

10. A pharmaceutical composition comprising a compound of formula (I) as defined in claim 1, or a pharmaceutically acceptable salt of solvate thereof, and one or more pharmaceutically acceptable excipient(s).

11. A method of treating pain in a mammal comprising administering to the mammal an effective amount of a compound of formula (I) as defined in claim 1, or a pharmaceutically acceptable salt or solvate thereof.

12. A compound of formula (I) according to claim 2 wherein each R³ is independently selected from halogen, (C₁-C₆)alkyl, (C₁-C₆)alkoxy, halo(C₁-C₆)alkyl, halo(C₁-C₆)alkoxy and halo(C₁-C₆)alkylthio.

13. A compound of formula (I) according to claim 2 wherein R¹ is hydrogen.

14. A compound of formula (I) according to claim 3 wherein R¹ is hydrogen.

15. A compound of formula (I) according to claim 12 wherein R¹ is hydrogen.

16. A compound formula (I) according to claim 2 wherein R⁴ and R^{4a}, are both hydrogen.

17. A compound formula (I) according to claim 3 wherein R⁴ and R^{4a} are both hydrogen.

18. A compound formula (I) according to claim 4 wherein R⁴ and R^{4a}, are both hydrogen.

19. A compound formula (I) according to claim 12 wherein R⁴ and R^{4a} are both hydrogen.

20. A compound formula (I) according to claim 13 wherein R⁴ and R^{4a}, are both hydrogen.

21. A compound formula (I) according to claim 14 wherein R⁴ and R^{4a} are both hydrogen.

22. A compound formula (I) according to claim 15 wherein R⁴ and R^{4a}, are both hydrogen.

23. A pharmaceutical composition comprising a compound of formula (I) as defined in claim 6, or a pharmaceutically acceptable salt of solvate thereof, and one or more pharmaceutically acceptable excipient(s).

24. A method of treating pain in a mammal comprising administering an effective amount of a compound of formula (I) as defined in claim 6, or a pharmaceutically acceptable salt or solvate thereof.

25. The method of claim 11 wherein the mammal is a human.

26. The method of claim 24 wherein the mammal is a human.
